(19) United States
(12) Patent Application Publication
(10) Pub. No.: US 2012/0209247 A1
(43) Pub. Date: Aug. 16, 2012

(54) Safe Disposable Injector with Changeable and Automatically Retractable Needle

(75) Inventor: Zhong FENG, Wuxi (CN)

(73) Assignee: WUXI YUSHOU MEDICAL APPLICANCE LTD.,, Wuxi (CN)

(21) Appl. No.: 13/364,151

(22) Filed: Feb. 1, 2012

Related U.S. Application Data
(63) Continuation-in-part of application No. 13/380,463, filed on Dec. 22, 2011.

(30) Foreign Application Priority Data
Jul. 15, 2009 (CN) 200910164711.1

(51) Int. Cl. A61M 5/32

(52) U.S. Cl. 604/506; 604/110

(57) ABSTRACT

A safe disposable injector with a changeable and automatically retractable needle includes a needle cylinder (1) and a plunger (2), and also includes a medicament-dispensing needle unit (3) and an injecting needle unit (4). The medicament-dispensing needle unit (3) and the injecting needle unit (4) are changed according to the requirement in use. The present invention will prevent harmful effects caused by needle deformation and variation of smoothness and sharpness during the injection to the human body. The operation of the present invention is the same as the convention injection operation. The present invention is convenient to use and reliable to operate. After injection, the injector needle can be retracted by an automatic retraction mechanism to avoid the accidental hurt to medical staff and patients by a polluted needle tip. The present invention is suitable for automatic production on the assembly line.

### CROSS REFERENCE OF RELATED APPLICATION

This is a Continuation-In-Part application that claims the benefit of priority under 35 U.S.C. §119 to a non-provisional application, application Ser. No. 13/380,463, filed Dec. 22, 2011.

### BACKGROUND OF THE PRESENT INVENTION

1. Field of Invention

The present invention relates to an injector, and more particular to a safe disposable injector, wherein the injecting needle is changeable and retractable after use.

2. Description of Related Arts

It is common practice for a disposable syringe to administer injections and vaccinations since the disposable syringe is a one-time-use device for preventing infectious diseases and nosocomial cross-infection. Such disposable syringe, such as syringe with auto-destroy needle, syringe with manual retractable needle, or syringe with automatic retractable needle, are generally of the disposable nature and are normally discarded after a single use. However, the disposable syringe has complicated structural configuration and lacks of necessary operational function. For example, when preparing dry powder injection, the syringe is used for injecting solvent into a sealed bottle in order to dissolve the dry powder in liquid form. In particular, the needle must be penetrated through the bottle stopper in order to inject the solvent into the bottle. In other words, the needle will be bent or deformed during the penetration. In addition, the tip of the needle will be blunt and the smoothness of the needle body will be substantially reduced after the penetration. Therefore, the syringe must be discarded after the preparation of the dry powder injection since the needle cannot be re-used to administer injection for human body. In fact, the syringe is prohibited for being re-used after preparation of the dry powder injection in many countries. Therefore, the physician must use one disposable syringe to prepare the dry powder injection and another one to administer injection. Accordingly, there is no syringe with changeable needle in the market, especially for the retractable needle.

In addition, the conventional automatic retractable needle is built-in with the barrel of the syringe, wherein the needle is irreplaceably fixed to the barrel in order to retract the needle back to the barrel via the automatic retractable needle. Accordingly, the structure of the automatic retractable needle is relatively complicated because the retractable mechanism must be built-in at the barrel, wherein the manufacturing cost of the automatic retractable needle is relatively high because of the complicated structural configuration of the retractable mechanism. In addition, when the syringe is being mishandled, such as the accidentally actuation of the plunger, the needle will be retracted before it is used.

### SUMMARY OF THE PRESENT INVENTION

The invention is advantageous in that it provides a safety disposable injector, which solves the existing problems of the syringe, wherein the needle is changeable and automatically retractable after use. The present invention is convenient to use and reliable to operate. After injection, the injector needle can be retracted by an automatic retraction mechanism to avoid an injury by accidental needle sticks as serious health hazard and danger of exposure to fatal blood-transmitted viruses.

Another advantage of the invention is to provide a safety disposable injector, wherein the automatic retraction mechanism is provided at the injecting needle unit to prevent the malfunction of the automatic retraction mechanism.

Another advantage of the invention is to provide a safety disposable injector, wherein the automatic retraction mechanism is provided at the injecting needle unit, such that the injecting needle unit is adapted for incorporating with any existing barrel in order to provide a safety and disposable feature of the injector.

Another advantage of the invention is to provide a safety disposable injector, which does not involve complicated or expensive mechanical components so as to minimize the manufacturing cost of the present invention.

Another advantage of the invention is to provide a safety disposable injector, wherein no expensive or complicated structure is required to employ in the present invention in order to achieve the above mentioned objects. Therefore, the present invention successfully provides an economic and efficient solution for providing an exchangeable and disposable needle unit for any syringe having a barrel structure.

Additional advantages and features of the invention will become apparent from the description which follows, and may be realized by means of the instrumentalities and combinations particular point out in the appended claims.

According to the present invention, the foregoing and other objects and advantages are attained by a safety disposable injector comprising a barrel and a plunger slidably coupling within the barrel in a reciprocal manner.

In accordance with another aspect of the invention, the present invention comprises a safety disposable injector comprising a barrel having a chamber, and a plunger slidably coupled within the chamber of the barrel in a reciprocal manner, wherein the plunger has a tubular structure and comprises a plunger stopper provided at a top end of the plunger.

The safety disposable injector further comprises a medicament-dispensing needle unit and an injecting needle unit selectively coupled with the barrel in sealed manner.

The medicament-dispensing needle unit comprises a medicament-dispensing needle body and a medicament-dispensing needle base, wherein the medicament-dispensing needle body is upwardly extended from the medicament-dispensing needle base.

The injecting needle unit comprises an injecting needle body, an injecting needle base, a base sleeve, a sealing element, a compression spring, and a spring seat, wherein the base sleeve, which is a tubular member, comprises a retention protrusion protruded from an inner wall of the base sleeve at a bottom end thereof, wherein the sealing element is supported within the base sleeve by the retention protrusion so as to retain the sealing element at the bottom end of the base sleeve, wherein the injecting needle base is held within an interior cavity of the base sleeve, wherein the spring seat is coupled at a top end of the base sleeve, wherein the sealing element is sealed and retained between the bottom end of the base sleeve and the bottom end of the injecting needle base to provide a sealing effect therebetween and to apply a frictional holding force against the injecting needle base to withstand a compression force of the compression spring.

The safety disposable injector further comprises a sealer element, such as an O-shaped sealing ring, provided at the barrel to seal with the medicament-dispensing needle unit and the injecting needle unit when the medicament-dispensing needle unit or the injecting needle unit is coupled with the barrel.

A top opening end portion of the barrel has an inner diameter gradually reducing toward the bottom end thereof to form an inner conical opening at an inner surface of the barrel. Correspondingly, each of the medicament-dispensing needle unit and the injecting needle unit further has an outer conical platform formed at an outer circumferential surface of the bottom end of each of the medicament-dispensing needle unit and the injecting needle unit to seal and engage with the inner conical opening of the barrel.

The taper of the inner conical opening of the barrel is larger than the taper of each of the outer conical platform of the medicament-dispensing needle unit and the injecting needle unit.

The medicament-dispensing needle base further has a handling wing, a rhombus cross section (non-circular cross section), or knurled outer surface at the outer surface of the medicament-dispensing needle base for enabling the operator to hold and turn the medicament-dispensing needle base to attach to or detach from the barrel.

The injecting needle unit further comprises an injecting needle protection sleeve for covering the injecting needle unit, wherein the injecting needle protection sleeve is coupled with the base sleeve to enclose the injecting needle body within the injecting needle protection sleeve, wherein the injecting needle unit further comprises an inner tongue-groove structure provided at an inner surface of the injecting needle protection sleeve and an outer tongue-groove structure provided at an outer surface of the base sleeve to engage with the inner tongue-groove structure.

The injecting needle protection sleeve further has a handling wing, a rhombus cross section (non-circular cross section), or knurled outer surface for enabling the operator to hold and turn the injection needle unit to attach to the barrel.

The injecting needle unit further comprises an inner ratchet provided at an inner surface of the barrel and an outer ratchet provided at an outer surface of the base sleeve wherein when the base sleeve is rotatably coupled with the barrel, the inner ratchet is engaged with the outer ratchet.

The tip of the medicament-dispensing needle body is configured as one of a slanted tip and a conical tip with a side aperture at a circumferential surface of a tip portion.

The barrel, the plunger, the medicament-dispensing needle base, the injecting needle base, base sleeve, and the spring seat are made of plastic.

The medicament-dispensing needle body and the medicament-dispensing needle base of the medicament-dispensing needle unit are formed in a one piece integrated structure by mold injection.

The barrel is made of transparent plastic material.

In accordance with another aspect of the invention, the present invention further provides a method of using a safety disposable injector, which comprises the following steps.

(A) Detachably couple an injection needle unit with a barrel.

(B) Actuate a plunger to inject fluid within the barrel through the injecting needle body.

(C) Push the plunger toward the injection needle unit until the plunger actuates the automatic retractable mechanism.

(D) Automatically retract the injecting needle body into the barrel.

Accordingly, the present invention provides the advantages as follows:

(1) According to the need of the injection, the operator is able to selectively interchange between the medicament-dispensing needle unit and the injecting needle unit to couple with the barrel. After the preparation of the liquid by the medicament-dispensing needle unit, the injecting needle unit to couple with the barrel to administer injections. Therefore, the sharpness of the injecting needle and the smoothness of the injecting needle will always be maintained at the best condition.

(2) The operation of the present invention is the same as the convention injection operation that the conventional operation habit remains basically unchanged in the present invention. The present invention is convenient to use and reliable to operate. After injection, the injector needle can be retracted by an automatic retraction mechanism to avoid the accidental hurt to medical staff and patients by a polluted needle tip.

(3) The present invention has a simple structural configuration and is suitable for automatic production on the assembly line, so that the present invention is capable of maintaining a high quality and reducing the manufacturing cost.

(4) As a result, the present invention will substantially reduce the unnecessary waste of material by interchanging the medicament-dispensing needle unit and the injecting needle unit, such that present invention is considered as an environmental friendly product.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of a safety disposable injector with medicament-dispensing needle unit according to a preferred embodiment of the present invention, illustrating the wing-type medicament-dispensing needle unit.

FIG. 2 is a sectional view of the safety disposable injector with medicament-dispensing needle unit according to the above preferred embodiment of the present invention, illustrating the rhombus-type medicament-dispensing needle unit.

FIG. 3 is a sectional view of the safety disposable injector with injection needle unit according to the above preferred embodiment of the present invention.

FIG. 4 is a sectional view of the safety disposable injector with injection needle unit according to the above preferred embodiment of the present invention, illustrating the injection needled unit being retracted.

FIG. 5 is a sectional view of the injection needle unit of the safety disposable injector according to the above preferred embodiment of the present invention.

FIG. 6 is a sectional view of the medicament-dispensing needle unit of the safety disposable injector according to the above preferred embodiment of the present invention.

FIG. 7 is a sectional view of the injection needle unit of the safety disposable injector according to the above preferred embodiment of the present invention, illustrating the rhombus-type injection needle unit.

FIG. 8 is a sectional view of the medicament-dispensing needle unit of the safety disposable injector according to the above preferred embodiment of the present invention, illustrating the rhombus-type medicament-dispensing needle unit.

FIG. 9 illustrates a sealing structure of the barrel of the safety disposable injector with injection needle according to the above preferred embodiment of the present invention.

FIG. 10 illustrates an automatic retraction mechanism of the safety disposable injector with injection needle according to the above preferred embodiment of the present invention.

### DETAILED DESCRIPTION OF THE PREFERRED EMBODIMENT

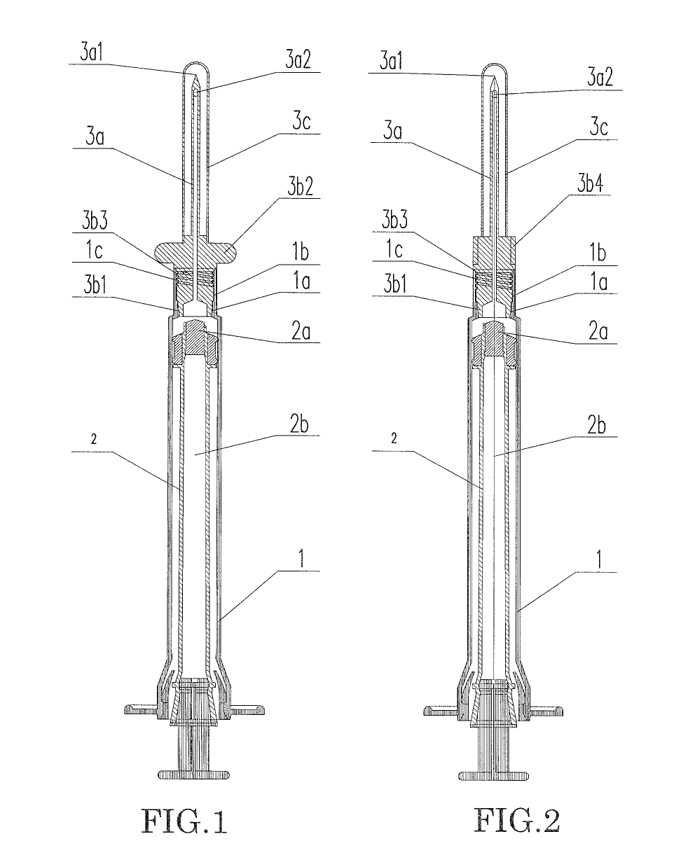

Referring to FIGS. 1 to 6 of the drawings, a safety disposable injector, such as a disposable syringe, according to a preferred embodiment of the present invention is illustrated, wherein the safety disposable injector comprises a barrel 1 having a chamber, and a plunger 2 slidably coupling within the chamber of the barrel 1 in a reciprocal manner.

The safety disposable injector further comprises a medicament-dispensing needle unit 3 and an injecting needle unit 4 selectively coupling with the barrel 1. According to the preferred embodiment, the medicament-dispensing needle unit 3 comprises a medicament-dispensing needle body 3a and a medicament-dispensing needle base 3b, wherein the medicament-dispensing needle body 3a is fixed and upwardly extended from the medicament-dispensing needle base 3b. Alternatively, the medicament-dispensing needle body 3a and the medicament-dispensing needle base 3b of the medicament-dispensing needle unit 3 can be formed in a one piece integrated structure by mold injection.

The injecting needle unit 4 comprises an injecting needle body 4a and a built-in automatic retraction mechanism which comprises an injecting needle base 4b, a base sleeve 4c, a sealing element 4f which is preferably a sealing O-ring, a compression spring 4d, and a spring seat 4e. The injecting needle body 4a is upwardly and coaxially extended from the injecting needle base 4b that a bottom portion of the injecting needle body 4a is held by the injecting needle base 4b. The base sleeve 4c, which is a tubular member, comprises a retention protrusion 4c1 protruded from an inner wall of the base sleeve 4c at a bottom end thereof, wherein the sealing element 4f is supported within the base sleeve 4c by the retention protrusion 4c1 so as to retain the sealing element 4f at the bottom end of the base sleeve 4c. The injecting needle base 4b is held within an interior cavity of the base sleeve 4c to define a gap therebetween. The spring seat 4e is coupled at a top end of the base sleeve 4c, wherein the spring seat 4e can be fixed to the base sleeve 4c by clamp-engagement, rotary-engagement, integrated structure or the like. The injecting needle body 4a is extended from the interior cavity of the base sleeve 4c to an exterior of the injecting needle base 4b through the spring seat 4e. The compression spring 4d is coaxially coupled with the injecting needle base 4b and is retained between the spring seat 4e and a bottom end of the injecting needle base 4b. Accordingly, the top end of the compression spring 4d is biased against the spring seat 4a while the bottom end of the compression spring 4d is biased against the bottom end of the injecting needle base 4b such that the compression spring 4d will apply a compression spring force against the injecting needle base 4b. In addition, the sealing element 4f is sealed and retained between the bottom end of the base sleeve 4c at the retention protrusion 4c1 thereof and the bottom end of the injecting needle base 4b to provide a sealing effect therebetween and to apply a frictional holding force against the injecting needle base 4b so as to withstand the compression force of the compression spring 4d. In other words, the sealing element 4f is provided at the gap between the injecting needle base 4b and the base sleeve 4c. Accordingly, the frictional holding force of the sealing element 4f equals to or greater than the compression force of the compression spring 4d, such that the injection needle base 4b will be retained in position and the compression spring 4d is retained at a compressed state. In other words, the injection needle base 4b is held within the base sleeve 4c via the sealing element 4f.

The barrel 1 has an inner threaded portion 1c. Correspondingly, the medicament-dispensing needle unit 3 further has an outer threaded portion 3b3 while the injecting needle unit 4 further has an outer threaded portion 4c5. According to the operation of the present invention, the medicament-dispensing needle unit 3 and the injecting needle unit 4 can be selectively coupled with the barrel 1 preferably by rotatably engaging the outer threaded portion 3b3 of the medicament-dispensing needle unit 3 to the inner threaded portion 1c of the barrel 1 or by rotatably engaging the outer threaded portion 4c5 of the injecting needle unit 4 to the inner threaded portion 1c of the barrel 1. It is appreciated that other engagements such as plug-in engagement or clip-on engagement can be used for selectively and detachably coupling one of the medicament-dispensing needle unit 3 and the injecting needle unit 4 with the barrel 1. It is worth mentioning that the top opening end portion of the barrel 1 has an inner diameter gradually reducing toward the bottom end thereof to form an inner conical opening 1a at an inner surface of the barrel 1. Correspondingly, each of the medicament-dispensing needle unit 3 and the injecting needle unit 4 further has an outer conical platform 3b1, 4c3 formed at an outer circumferential surface of the bottom end of each of the medicament-dispensing needle unit 3 and the injecting needle unit 4. It is worth mentioning that each of the medicament-dispensing needle unit 3 and the injecting needle unit 4 can have a circular platform instead of the outer conical platform 3b1, 4c3. When the medicament-dispensing needle unit 3 or the injecting needle unit 4 is rotatably engaged with the barrel 1, the outer conical platform 3b1, 4c3 of the medicament-dispensing needle unit 3 or the injecting needle unit 4 will bias against the inner conical opening 1a of the barrel 1 so as to create a sealing effect between the outer conical platform 3b1, 4c3 of the medicament-dispensing needle unit 3/the injecting needle unit 4 and the inner conical opening 1a of the barrel 1. If the taper of the inner conical opening 1a of the barrel 1 is different from the taper of the outer conical platform 3b1, 4c3 of the medicament-dispensing needle unit 3 or the injecting needle unit 4, i.e. the taper of the inner conical opening 1a of the barrel 1 is larger than the taper of the outer conical platform 3b1, 4c3 of the medicament-dispensing needle unit 3 or the injecting needle unit 4, the sealing effect between the two surfaces will be achieved. For the best mode, when the taper of the inner conical opening 1a of the barrel 1 matches with the taper of the outer conical platform 3b1, 4c3 of the medicament-dispensing needle unit 3 or the injecting needle unit 4, the sealing effect between the surfaces will be maximized by increasing the contacting surface area. It is appreciated that an O-shaped sealing ring or other sealer element can be used for sealing between the two surfaces to further enhance the sealing effect therebetween.

In order to operate the present invention, the medicament-dispensing needle unit 3 is firstly coupled with the barrel 1 such that by slidably pulling the plunger 2, the solvent is sucked into the barrel 1 via the medicament-dispensing needle unit 3. Then, by inserting the medicament-dispensing needle unit 3 into a bottle containing the dry powder medication, the solvent is injected into a bottle containing the dry powder medication via the slidably pushing the plunger 2, so as to dissolve the dry powder medication to form a liquid medication. By slidably pulling the plunger 2, the liquid medication is withdrawn from the bottle to the barrel 1 through the medicament-dispensing needle unit 3. The medicament-dispensing needle unit 3 is detached from the barrel 1 and the injection needle unit 4 is then coupled with the barrel 1.

Accordingly, the safety disposable injector further comprises a locking means for locking the injection needle unit 4 with the barrel 1, wherein after the injection needle unit 4 is coupled to the barrel 1, the injection needle unit 4 cannot be detached from the barrel 1. The locking means comprises an outer ratchet 4c4 outwardly protruded from the outer circumferential surface of the injection needle unit 4, and correspondingly, an inner ratchet 1b protruded from the inner circumferential surface of the barrel 1, wherein when the injection needle unit 4 is rotatably engaged with the barrel 1 at one direction, the outer ratchet 4c4 is engaged with the inner ratchet 1b to prevent the injection needle unit 4 being reversely rotated back to an opposite direction. In other words, the injection needle unit 4 can only coupled with the barrel 1 but cannot detached from the barrel 1 once the injection needle unit 4 is coupled thereto. Once the injection needle unit 4 is coupled with the barrel 1, the plunger 2 is slidably pushed to administer injection through the injection needle unit 4.

Accordingly, the plunger 2, having a tubular structure, comprises a plunger body and a tubular plunger stopper 2a coupled at a top end of the plunger body of plunger 2 and adapted to being pushed into the interior of the plunger 2 when a downward pushing force is applied at the plunger stopper 2a. When the plunger 2 is pushed toward the injection needle unit 4, the plunger stopper 2a of the plunger 2 will bias against the bottom end of the injection needle base 4b. When the pushing force is kept applying at the plunger body of the plunger 2, the plunger stopper 2a is forced and gradually pushed back into the interior of the plunger body of the plunger 2. At the mean time, the top end of the plunger body of the plunger 2 is aligned with the sealing element 4f and is slidably inserted into the gap between the bottom end of the base sleeve 4c and the bottom end of the injecting needle base 4b. When the pushing force is kept applying at the plunger body of the plunger 2, the top end of the plunger 2 will push against the sealing element 4f until the sealing element 4f is moved offset to its original position so as to dislocate the sealing element 4f. Therefore, the frictional holding force of the sealing element 4f will be released to free the engagement between the base sleeve 4c and the injecting needle base 4b. It is worth mentioning that all the liquid medication will be discharged from the barrel 1 at this position. Once the frictional holding force of the sealing element 4f is released, the compressed compression spring 4d will return back to its original form to apply the spring force against the injecting needle base 4b. Therefore, the injecting needle base 4b, including the injecting needle body 4a, will be pushed into the interior of the plunger body of the plunger 2 through the sealing element 4f so as to retract the injecting needle body 4a into the barrel 1. It is worth mentioning that the injecting needle base 4b will also push the plunger stopper 2a together into the interior of the plunger body the plunger 2. In other words, when the injecting needle body 4a is rapidly retracted into the barrel 1, i.e. also the interior of the plunger 2, via the spring force, the injecting needle body 4a will be pulled back from the patient skin to complete the injection process. It is worth mentioning that the length of the plunger 2 is longer than the length of the injecting needle body 4a such that the entire injecting needle body 4a is retracted into the interior of the plunger 2. Therefore, no protection cap is required for covering the top opening of the barrel 1 to prevent the tip of the used injecting needle body 4a, so as to avoid an injury by accidental needle sticks. In addition, the used injection needle unit 4 cannot be re-assembled back to the barrel 1 to prevent the repeated use of the injection needle unit 4 for ensuring the injection needle unit 4 as a one-time use device.

### Embodiment 2

Embodiment 2 is a modification of the above embodiment, wherein the safety disposable injector further comprises a medicament-dispensing needle protection sleeve 3c for covering the medicament-dispensing needle unit 3. Accordingly, the medicament-dispensing needle protection sleeve 3c is coupled with the medicament-dispensing needle base 3b to enclose the medicament-dispensing needle body 3a within the medicament-dispensing needle protection sleeve 3c. The medicament-dispensing needle base 3b further has a handling wing 3b2, a rhombus cross section (non-circular cross section) 3b4, or knurled outer surface at the outer surface of the medicament-dispensing needle base 3b for enabling the operator to hold and turn the medicament-dispensing needle base 3b to attach to or detach from the barrel 1. Accordingly, the tip of the medicament-dispensing needle body 3a can be formed as a slanted tip or a conical tip 3a1 with a side aperture 3a2 at the circumferential surface of the tip portion.

### Embodiment 3

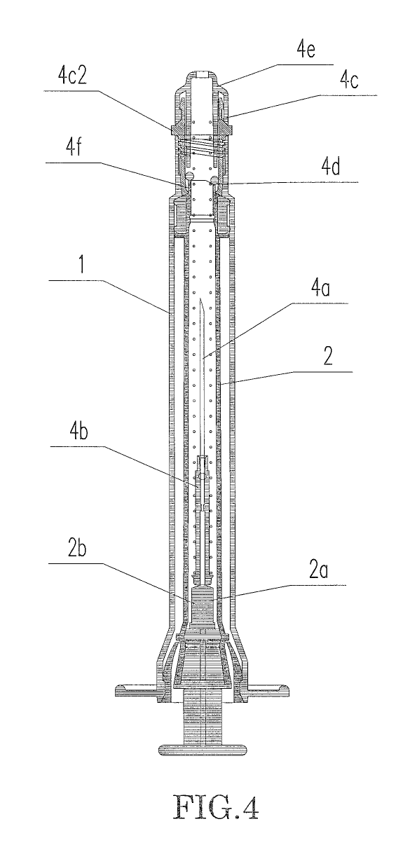

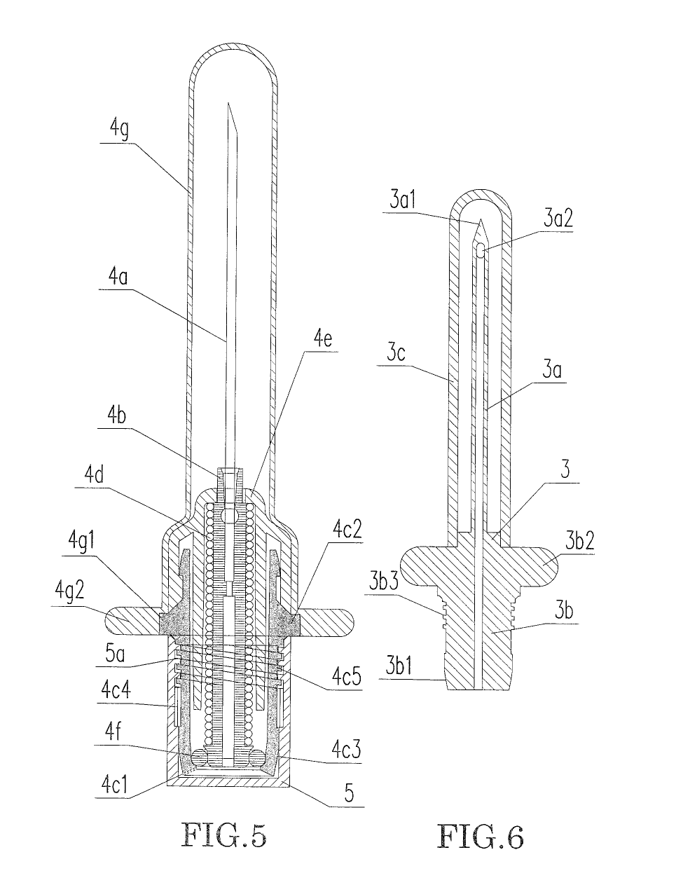

Embodiment 3 is another modification of the above embodiment, as shown in FIGS. 4 and 5, wherein the safety disposable injector further comprises an injecting needle protection sleeve 4g for covering the injecting needle unit 4. Accordingly, the injecting needle protection sleeve 4g is coupled with the base sleeve 4c to enclose the injecting needle body 4a within the injecting needle protection sleeve 4g. The injecting needle unit 4 further comprises an inner tongue-groove structure 4g1 provided at an inner surface of the injecting needle protection sleeve 4g and an outer tongue-groove structure 4c2 provided at an outer surface of the base sleeve 4c. Therefore, when the injecting needle protection sleeve 4g is coupled with the base sleeve 4c to enclose the injecting needle body 4a, the inner tongue-groove structure 4g1 is interlocked with the outer tongue-groove structure 4c2. The injecting needle protection sleeve 4g further has a handling wing 4g2, a rhombus cross section (non-circular cross section) 4g3, or knurled outer surface for enabling the operator to hold and turn the injection needle unit 4 to attach to the barrel 1. It is worth mentioning that before the injection needle unit 4 is attached to the barrel 1, the base sleeve 4c is covered and protected by a base cover 5. The base cover 5 can be removed from the base sleeve 4c in order for the injection needle unit 4 attaching to the barrel 1. Preferably, the base cover 5 has an inner threaded portion 5a rotatably and detachably coupled with the base sleeve 4c.

### Embodiment 4

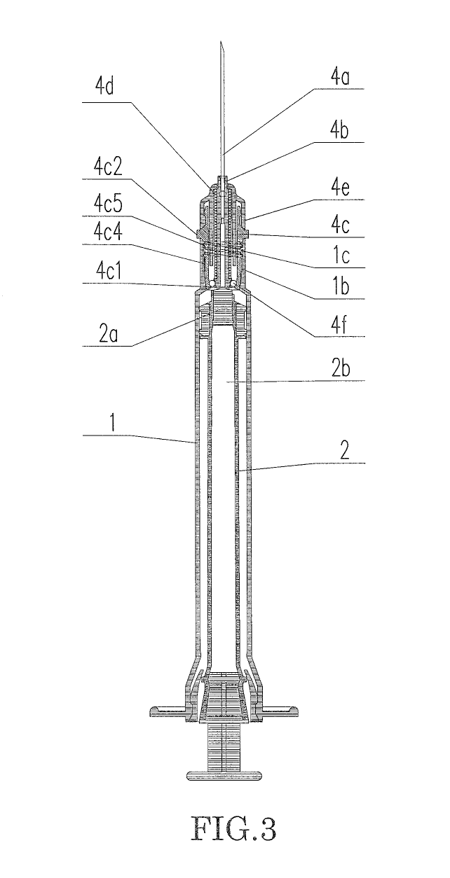

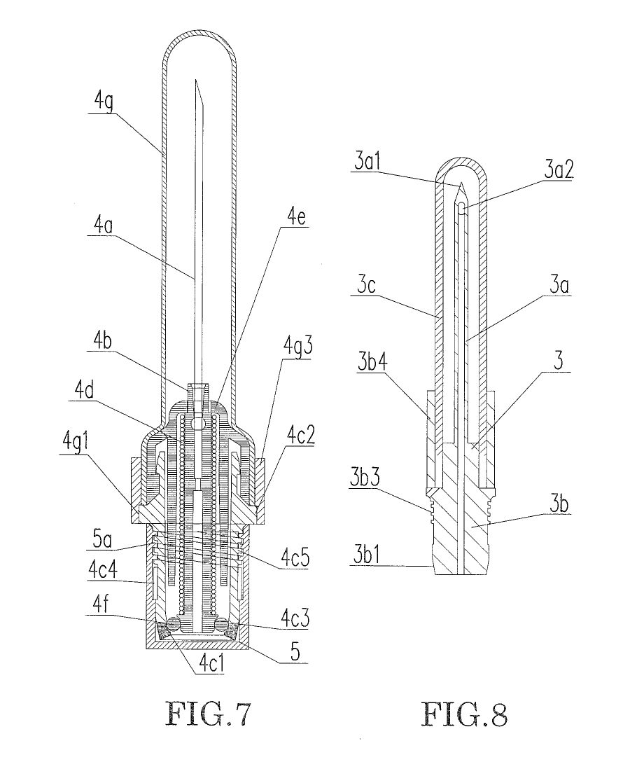

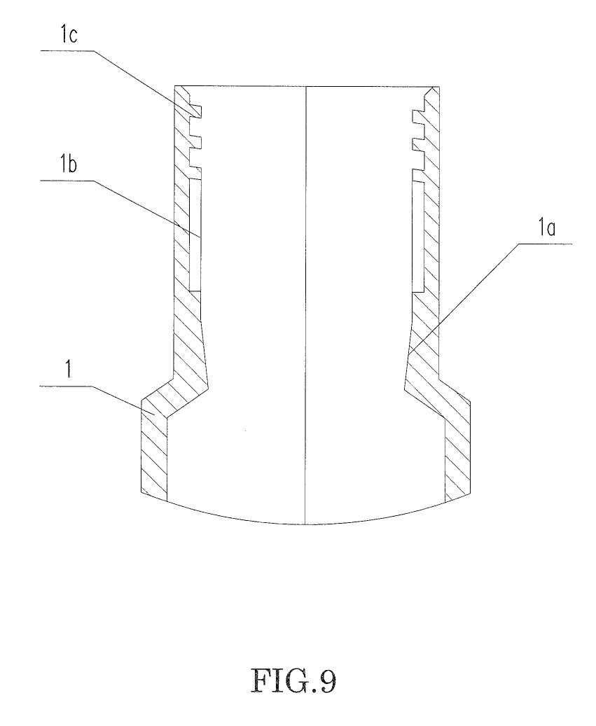

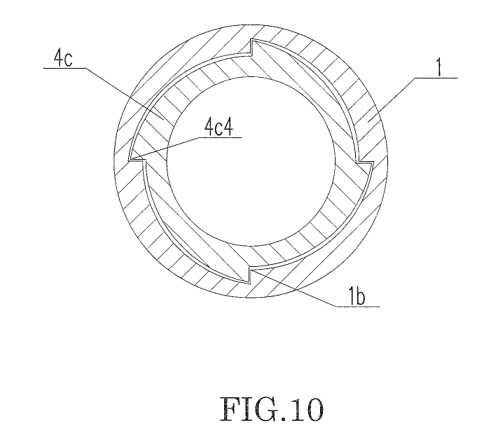

Embodiment 4 is another modification of the above first and third embodiments, as shown in FIGS. 3, 4, 7 and 9, wherein the automatic retraction mechanism of the injecting needle unit 4 further comprises an inner ratchet 1b provided at the inner surface of the barrel 1 and an outer ratchet 4c4 provided at the outer surface of the base sleeve 4c. When the base sleeve 4c is rotatably coupled with the barrel 1, the inner ratchet 1b is engaged with the outer ratchet 4c4. Therefore, the base sleeve 4c can only rotate at one direction to couple with the barrel 1, as shown in FIG. 10, so as to prevent the base sleeve 4c being detached from the barrel 1 by rotating the base sleeve 4c at the reverse direction. In other words, the present invention prevents the injecting needle unit 4 being re-used by disassembling the injecting needle unit 4 from the barrel 1 after use. Therefore, the present invention ensures the injecting needle unit 4 being used for one single time only.

According to the preferred embodiment, a method of using the safety disposable injector of the present invention comprises the following steps.

(1) Detachably couple the injection needle unit 4 with the barrel 1. Preferably, the injection needle unit 4 is rotatably coupled with the barrel 1. It is worth mentioning that once the injection needle unit 4 is coupled with the barrel 1, the injection needle unit 4 cannot be detached from the barrel 1 via the locking means.

(2) Actuate the plunger 2 to inject fluid within the barrel 1 through the injecting needle body 4. In other words, when the plunger 2 is pushed within the barrel 1, the fluid is rejected through the injecting needle body 4.

(3) Push the plunger 2 toward the injection needle unit 4 until the plunger 2 actuates the automatic retractable mechanism. Accordingly, when the pushing force is kept applying at the plunger body of the plunger 2, the plunger stopper 2a is forced and gradually pushed back into the interior of the plunger body of the plunger 2. When the pushing force is kept applying at the plunger body of the plunger 2, the top end of the plunger body of the plunger 2 will fit into the gap the bottom end of the base sleeve 4c and the bottom end of the injecting needle base 4b. Therefore, the top end of the plunger body of the plunger 2 will dislocate the sealing element 4f.

(4) Automatically retract the injecting needle body 4a into the barrel. Accordingly, once the sealing element 4f is dislocated, the injecting needle body 4a will be pushed into the interior of the plunger body of the plunger 2 by the spring force of the compression spring 4d.

Accordingly, before the injection needle unit 4 is coupled with the barrel 1, the present invention further provides the following pre-steps to prepare the fluid in the barrel 1.

(A) Detachably couple the medicament-dispensing needle unit 3 with the barrel 1. Preferably, the medicament-dispensing needle unit 3 is rotatably coupled with the barrel 1. It is worth mentioning that since the automatic retractable mechanism is built-in with the injection needle unit 4 but not the barrel 1, the medicament-dispensing needle unit 3 will not be retracted into the barrel 1. In addition, since the medicament-dispensing needle unit 3 does not contain the locking means, the medicament-dispensing needle unit 3 can be detached from the barrel 1.

(B) Actuate the plunger 2 to draw the fluid into the barrel 1 through the medicament-dispensing needle unit 3. Accordingly, the plunger 2 is pulled away from the medicament-dispensing needle unit 3 for creating a suction effect within the barrel 1 in order to suck the fluid into the barrel 1. It is worth mentioning that the solvent is sucked into the barrel 1 via the medicament-dispensing needle unit 3 and is injected into the bottle containing the dry powder medication via the medicament-dispensing needle unit 3, so as to dissolve the dry powder medication to form a fluid as the liquid medication.

(C) Detach the medicament-dispensing needle unit 3 from the barrel 1. Accordingly, the injection needle unit 4 can be coupled with the barrel 1 once the medicament-dispensing needle unit 3 is detached from the barrel 1. It is worth mentioning that a cap can be coupled at the top opening of the barrel 1 at the time after the medicament-dispensing needle unit 3 is detached from the barrel 1 and before the injection needle unit 4 is coupled with the barrel 1.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A safety disposable injector, comprising:
a barrel having a chamber;
a plunger slidably coupled within said chamber of said barrel in a reciprocal manner; and
an injecting needle unit, which is detachably coupled with said barrel, comprising:
an injecting needle body; and
an automatic retractable mechanism coupled with said injecting needle body, wherein after said injecting needle unit is detachably coupled with said barrel, said plunger is pushed toward said injecting needle body to actuate said automatic retractable mechanism so as to retract said injecting needle body into said barrel.

2. The safety disposable injector, as recited in claim 1, wherein said plunger has a tubular body and a plunger stopper provided at a top end of said plunger body and arranged in such a manner that when said tubular body is pushed within said barrel to actuate said automatic retractable mechanism, said plunger stopper and said injecting needle body are retracted into said tubular body.

3. The safety disposable injector, as recited in claim 1, wherein said automatic retractable mechanism comprises an injecting needle base coaxially holding a lower end portion of said injection needle body in position, a base sleeve detachably coupled with said barrel and coaxially receiving said injecting needle base in said base sleeve to define a gap between said injection needle base and said base sleeve, a compression spring coaxially coupled with said injection needle body for applying a pushing force against said injection needle body toward said barrel, and a sealing element coaxially coupled between said injecting needle base and said base sleeve at said gap to retain said injecting needle base within said base sleeve, wherein when said plunger is pushed to dislocate said sealing element, said injection needle body is downwardly pushed by said compression spring so as to retract said injecting needle body into said barrel.

4. The safety disposable injector, as recited in claim 2, wherein said automatic retractable mechanism comprises an injecting needle base coaxially holding a lower end portion of said injection needle body in position, a base sleeve detachably coupled with said barrel and coaxially receiving said injecting needle base in said base sleeve to define a gap between said injection needle base and said base sleeve, a compression spring coaxially coupled with said injection needle body for applying a pushing force against said injection needle body toward said barrel, and a sealing element coaxially coupled between said injecting needle base and said base sleeve at said gap to retain said injecting needle base within said base sleeve, wherein when said plunger is pushed to dislocate said sealing element, said injection needle body is downwardly pushed by said compression spring so as to retract said injecting needle body into said barrel.

5. The safety disposable injector, as recited in claim 3, wherein said sealing element is a sealing ring coaxially coupled and sealed between said injecting needle base and said base sleeve.

6. The safety disposable injector, as recited in claim 4, wherein said sealing element is a sealing ring coaxially coupled and sealed between said injecting needle base and said base sleeve.

7. The safety disposable injector, as recited in claim 4, wherein said base sleeve comprises a retention protrusion protruded from an inner wall of said base sleeve at a bottom end thereof, wherein said sealing element is supported within said base sleeve by the retention protrusion to retain said sealing element at said bottom end of said base sleeve.

8. The safety disposable injector, as recited in claim 6, wherein said base sleeve comprises a retention protrusion protruded from an inner wall of said base sleeve at a bottom end thereof, wherein said sealing element is supported within said base sleeve by the retention protrusion to retain said sealing element at said bottom end of said base sleeve.

9. The safety disposable injector, as recited in claim 4, wherein said top end of said plunger body is pushed into said gap to dislocate said sealing element after said plunger stopper is pushed into said plunger body.

10. The safety disposable injector, as recited in claim 6, wherein said top end of said plunger body is pushed into said gap to dislocate said sealing element after said plunger stopper is pushed into said plunger body.

11. The safety disposable injector, as recited in claim 8, wherein said top end of said plunger body is pushed into said gap to dislocate said sealing element after said plunger stopper is pushed into said plunger body.

12. The safety disposable injector, as recited in claim 1, further comprising a locking means for locking said injection needle unit with said barrel, wherein said locking means comprises an outer ratchet outwardly protruded from an outer circumferential surface of said injection needle unit, and an inner ratchet protruded from an inner circumferential surface of said barrel, such that after said injection needle unit is coupled to said barrel, said outer ratchet is engaged with said inner ratchet to prevent said injection needle unit being detached from said barrel.

13. The safety disposable injector, as recited in claim 4, further comprising a locking means for locking said injection needle unit with said barrel, wherein said locking means comprises an outer ratchet outwardly protruded from an outer circumferential surface of said injection needle unit, and an inner ratchet protruded from an inner circumferential surface of said barrel, such that after said injection needle unit is coupled to said barrel, said outer ratchet is engaged with said inner ratchet to prevent said injection needle unit being detached from said barrel.

14. The safety disposable injector, as recited in claim 11, further comprising a locking means for locking said injection needle unit with said barrel, wherein said locking means comprises an outer ratchet outwardly protruded from an outer circumferential surface of said injection needle unit, and an inner ratchet protruded from an inner circumferential surface of said barrel, such that after said injection needle unit is coupled to said barrel, said outer ratchet is engaged with said inner ratchet to prevent said injection needle unit being detached from said barrel.

15. A method of using a safety disposable injector, comprising the steps of:
(a) detachably coupling an injection needle unit with a barrel, wherein said injection needle unit comprises an injecting needle body and an automatic retractable mechanism built-in with said injecting needle body;
(b) actuating a plunger to inject fluid within said barrel through said injecting needle body;
(c) keep pushing said plunger toward said injection needle unit until said plunger actuates said automatic retractable mechanism; and
(d) automatically retracting said injecting needle body into said barrel.

16. The method, as recited in claim 15, wherein said automatic retractable mechanism comprises an injecting needle base coaxially holding a lower end portion of said injection needle body in position, a base sleeve detachably coupled with said barrel and coaxially receiving said injecting needle base in said base sleeve to define a gap between said injection needle base and said base sleeve, a compression spring coaxially coupled with said injection needle body for applying a pushing force against said injection needle body toward said barrel, and a sealing element coaxially coupled between said injecting needle base and said base sleeve at said gap to retain said injecting needle base within said base sleeve, wherein, in the step (c), said plunger is pushed to dislocate said sealing element, such that said injection needle body is downwardly pushed by said compression spring to retract said injecting needle body into said barrel.

17. The method, as recited in claim 16, wherein said plunger has a tubular body and a plunger stopper provided at a top end of said plunger body, wherein, in the step (c), when said tubular body is pushed within said barrel to actuate said automatic retractable mechanism, said plunger stopper and said injecting needle body are retracted into said tubular body.

18. The method, as recited in claim 17, wherein said top end of said plunger body is pushed into said gap to dislocate said sealing element after said plunger stopper is pushed into said plunger body.

19. The method, as recited in claim 18, wherein said sealing element is a sealing ring coaxially coupled and sealed between said injecting needle base and said base sleeve.

20. The method, as recited in claim 15, wherein the step (a) further comprises a step of locking said injection needle unit with said barrel after said injection needle unit is coupled to said barrel to prevent said injection needle unit being detached from said barrel.

21. The method, as recited in claim 19, wherein the step (a) further comprises a step of locking said injection needle unit with said barrel after said injection needle unit is coupled to said barrel to prevent said injection needle unit being detached from said barrel.

22. The method, as recited in claim 15, before the step (a), further comprising the steps of:
detachably coupling a medicament-dispensing needle unit with said barrel;
actuating said plunger to draw said fluid into said barrel through said medicament-dispensing needle unit; and
detaching said medicament-dispensing needle unit from said barrel.

23. The method, as recited in claim 19, before the step (a), further comprising the steps of:
detachably coupling a medicament-dispensing needle unit with said barrel;
actuating said plunger to draw said fluid into said barrel through said medicament-dispensing needle unit; and
detaching said medicament-dispensing needle unit from said barrel.

24. The method, as recited in claim 21, before the step (a), further comprising the steps of:
detachably coupling a medicament-dispensing needle unit with said barrel;
actuating said plunger to draw said fluid into said barrel through said medicament-dispensing needle unit; and
detaching said medicament-dispensing needle unit from said barrel.
